(19) United States
(12) Patent Application Publication
(10) Pub. No.: US 2016/0113589 A1
(43) Pub. Date: Apr. 28, 2016

(54) BIOSIGNAL PROCESSING METHOD AND APPARATUS

(71) Applicant: SAMSUNG ELECTRONICS CO., LTD., Suwon-si (KR)

(72) Inventor: Youngzoon Yoon, Hwaseong-si (KR)

(73) Assignee: SAMSUNG ELECTRONICS CO., LTD., Suwon-si (KR)

(21) Appl. No.: 14/833,221

(22) Filed: Aug. 24, 2015

(30) Foreign Application Priority Data
Oct. 23, 2014 (KR) 10-2014-0144281

(51) Int. Cl. A61B 5/00; A61B 5/0295; A61B 5/021; A61B 5/02

(52) U.S. Cl. CPC A61B 5/7257; A61B 5/02028; A61B 5/7235; A61B 5/0295; A61B 5/6824; A61B 5/02108; A61B 5/02007; A61B 2562/02; A61B 2562/0219

(57) ABSTRACT

Biosignal processing method and apparatus are provided. The biosignal processing method includes: detecting a biosignal, which is generated by a movement of a heart existing in a second area of a subject, from a first area of the subject; generating of a biosignal waveform from the biosignal; determining a relative position of the first area with respect to the second area based on at least one of the biosignal waveform and a direction of the first area; and converting the biosignal waveform to a reference biosignal waveform based on the relative position.

### CROSS-REFERENCE TO RELATED APPLICATION

This application claims priority from Korean Patent Application No. 10-2014-0144281, filed on Oct. 23, 2014 in the Korean Intellectual Property Office, the disclosure of which is incorporated herein in its entirety by reference.

### BACKGROUND

1. Field

Apparatuses and methods consistent with exemplary embodiments relate to processing a biosignal.

2. Description of the Related Art

A blood pressure measurement result is used to evaluate a physical condition of an individual. Blood pressure monitors capable of measuring a blood pressure are widely used in medical institutions and at homes. Cuff-type blood pressure monitors measure a systolic blood pressure and a diastolic blood pressure while using a cuff to apply a pressure to an area through which arterial blood flows, so as to stop a blood flow, and gradually reduce a pressure.

The cuff-type blood pressure monitors are large in size and are inconvenient to carry. Hence, the cuff-type blood pressure monitors are inappropriate to monitor a continuous change in a blood pressure of an individual in real time. Therefore, extensive research has been conducted to develop cuffless blood pressure monitors.

The cuffless blood pressure monitors may use a correlation of blood pressure based on a time difference between electrocardiography (ECG) and photoplethysmography (PPG) using a pulse transit time (PTT) method, or may estimate a blood pressure by analyzing a PPG waveform alone. Since the PTT method needs to use the ECG, the PTT method is unsuitable as a continuous measurement method using a single band. In the method of estimating the blood pressure by analyzing the waveform of the PPG alone, the waveform of the PPG is greatly changed according to a difference between a position of a wrist and a position of a heart.

### SUMMARY

One or more exemplary embodiments provide methods and apparatuses for converting a biosignal waveform to a reference biosignal waveform when a relative position between a detection spot from which a biosignal is detected and a source that generates the biosignal is changed.

Further, one or more exemplary embodiments provide method and apparatuses for providing information on a biological condition of a subject by using a biosignal.

According to an aspect of an exemplary embodiment, there is provided a biosignal processing method including: detecting from a first area of a subject a biosignal, which is generated by a movement of a heart existing in a second area of the subject; generating a biosignal waveform from the biosignal; determining a relative position of the first area with respect to the second area by using at least one of the biosignal waveform and a direction of the first area; and converting the biosignal waveform to a reference biosignal waveform by using the relative position.

The converting may include: reading a transfer function corresponding to the relative position from metadata; and acquiring the reference biosignal waveform by applying the read transfer function to the biosignal waveform to convert the biosignal waveform to the reference biosignal waveform.

The transfer function may include a first transfer function of an amplitude part and a second transfer function of a phase part.

The first transfer function may be defined as an amplitude ratio between biosignal waveforms detected at different positions, and the second transfer function may be defined as a phase difference between the biosignal waveforms detected at the different positions.

The converting may include: dividing the biosignal waveform into an amplitude part and a phase part by using a discrete Fourier transform; and applying the first transfer function to the amplitude part, applying the second transfer function to the phase part, and acquiring the reference biosignal waveform by using a discrete Fourier transform.

The reference biosignal waveform may be a biosignal waveform at a reference position.

The reference position may be a position at which heights of the first area and the second area are equal to each other.

The biosignal may be a photoplethysmography (PPG) signal.

The direction of the first area may be detected by a direction sensor disposed in the first area.

The direction sensor may be a tilt sensor.

The determining of the relative position may include, when a single relative position is expected from the direction of the first area, determining the expected relative position as the relative position.

The determining of the relative position may include, when a plurality of relative positions are expected from the direction of the first area, determining one of the plurality of expected relative positions as the relative position.

The determining one of the plurality of expected relative positions as the relative position may include: extracting factors including at least two of an augmentation index, a minimum systolic time, and a reflect wave time; and comparing the extracted factors with factors corresponding to the reference biosignal wave.

The first area may be a wrist of the subject.

The biosignal processing method may further include estimating information on a biological condition of the subject by using the reference biosignal waveform.

The information on the biological condition of the subject may include at least one of blood pressure information and vascular compliance information.

According to an aspect of another exemplary embodiment, there is provided a biosignal processing apparatus including: a first sensor configured to detect a biosignal, which is generated by a movement of a heart existing in a first area of a subject, from a second area of the subject; and a processor configured to generate a biosignal waveform from the biosignal and convert the biosignal waveform to a reference biosignal waveform by using a relative position of the second area with respect to the first area.

The biosignal processing apparatus may further include a memory configured to store metadata in which a transfer function for converting the biosignal waveform to the reference biosignal waveform is defined for each position, wherein the processor is configured to read a transfer function corresponding to the relative position from the memory and acquires the reference biosignal waveform by applying the read transfer function to the biosignal waveform.

The reference biosignal waveform may be a biosignal waveform at a reference position.

The biosignal processing apparatus may further include a second sensor configured to detect a direction of the second area.

According to an aspect of another exemplary embodiment, there is provided a method of processing a biosignal measuring device including: detecting a biosignal from a detection point of the subject on which the biosignal measuring device is placed; generating a biosignal waveform from the biosignal; determining a relative position of the biosignal measuring device with respect to a reference point of the subject based on a tilt angle of the biosignal measuring device; and correcting the biosignal waveform based on the relative position.

The reference point may be located at the heart of the subject and the corrected biosignal waveform may indicate a blood pressure of the subject.

### BRIEF DESCRIPTION OF THE DRAWINGS

The above and/or other aspects will be more apparent by describing certain exemplary embodiments, with reference to the accompanying drawings, in which:

FIGS. 1A and 1B are conceptual diagrams of a wearable device worn on a wrist to process a biosignal, according to an exemplary embodiment;

FIG. 2 is a diagram describing an area of a wrist from which a wristwatch type or wristband type biosignal processing apparatus detects a biosignal, according to an exemplary embodiment;

FIG. 3 is a block diagram of a biosignal processing apparatus according to an exemplary embodiment;

FIG. 4 is a graph of a biosignal waveform according to an exemplary embodiment;

FIG. 5A is a diagram illustrating a change in positions of detection spots with respect to a heart;

FIG. 5B is a graph of biosignal waveforms measured at the respective detection spots of FIG. 5A;

FIG. 6A is a graph of a maximum systolic time and a reflect wave time with respect to a position of a detection spot, according to an exemplary embodiment;

FIG. 6B is a graph of an augmentation index with respect to a position of a detection spot, according to an exemplary embodiment;

FIG. 6C is a graph of a peak systolic velocity with respect to a position of a detection spot, according to an exemplary embodiment;

FIG. 7 is a graph of a blood pressure with respect to a position of a detection spot;

FIG. 8 is a diagram illustrating a direction of a sensor with respect to a position of a detection spot when a 1-axis horizontal sensor is worn, according to an exemplary embodiment;

FIGS. 9A, 9B, and 9C are reference diagrams describing a relative position of a detection spot and a detection result of a horizontal sensor, according to an exemplary embodiment;

FIGS. 10A and 10B are diagrams illustrating a change in a biosignal waveform with respect to a detection spot, according to an exemplary embodiment;

FIG. 11 is a block diagram of a processor of FIG. 3;

FIG. 12A is a graph of a first transfer function for a detection spot;

FIG. 12B is a graph of a second transfer function for a detection spot;

FIG. 13 is a flowchart of a biosignal processing method according to an exemplary embodiment;

FIG. 14 is a flowchart of a method of determining a relative position of a detection spot from a biosignal waveform, according to an exemplary embodiment;

FIG. 15 is a flowchart of a method of determining a relative position of a detection spot, according to another exemplary embodiment; and

FIG. 16 is a reference diagram describing a method by which a biosignal processing apparatus provides information on a blood pressure, according to another exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed.

It will be understood that when a layer, region, or component is referred to as being “formed on,” another layer, region, or component, it can be directly or indirectly formed on the other layer, region, or component. That is, for example, intervening layers, regions, or components may be present. In addition, the terms “unit” and “module” may refer to unit of processing at least one function or operation and the “unit” and “module” may be implemented by hardware, software, or a combination thereof.

It will be further understood that the terms “comprises” and/or “comprising” used herein specify the presence of stated features or components, but do not preclude the presence or addition of one or more other features or components.

It will be understood that, although the terms “first”, “second”, etc. may be used herein to describe various elements, these elements should not be limited by these terms. These terms are only used to distinguish one element from another.

The term “subject” used herein refers to an object from which a biological condition is to be measured and may include a human, an animal, or the like. The term “object” used herein refers to a part of a subject and refers to a source that generates a biosignal by a movement. For example, the object may be a heart. The term “biosignal” refers to a unique signal that is generated from a subject. Examples of the biosignal may include electrocardiogram (ECG), ballistocardiogram (BCG), photoplethysmograph (PPG), a brain wave, and electromyogram (EMG). In addition, the user may be a subject from which a biosignal is to be measured, but the user may be a medical expert having an ability to use the biosignal processing apparatus. That is, a user may be a broader concept than a subject.

A biosignal processing apparatus according to an exemplary embodiment may be a device capable of being carried by a subject. For example, the biosignal processing apparatus may be a wearable device. The biosignal processing apparatus may include a wristwatch type apparatus, a bracelet type apparatus, a ring type apparatus, or a headband type apparatus, each of which has a communication function and a data processing function. In the present exemplary embodiments, it is assumed that the biosignal processing apparatus is a wristwatch type or wristband type apparatus, but the present exemplary embodiments are not limited thereto.

In addition, the biosignal processing apparatus may be implemented using a single housing or a plurality of housings. In a case where the biosignal processing apparatus is implemented using a plurality of housings, a plurality of components may be connected to one another by wire or wireless. For example, the biosignal processing apparatus may be divided into a first apparatus that includes a sensor worn on a wrist of a subject to detect a biosignal, and a second apparatus that processes the detected biosignal.

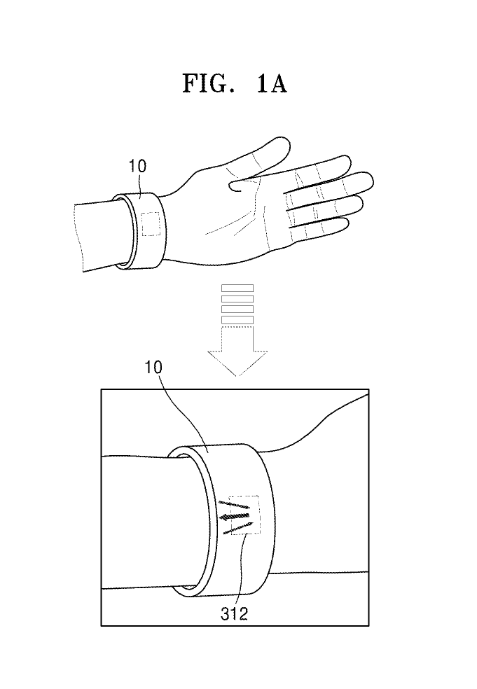

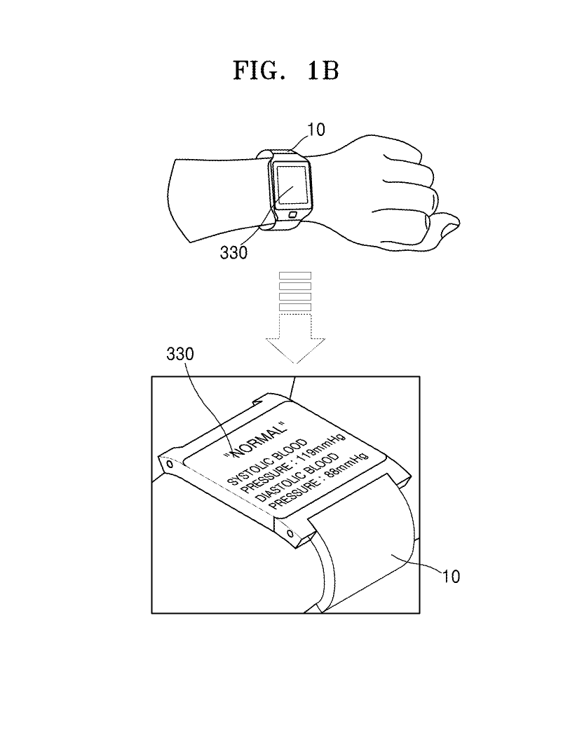

FIGS. 1A and 1B are conceptual diagrams of a wearable device worn on a wrist to process a biosignal, according to an exemplary embodiment. Referring to FIG. 1A, the biosignal processing apparatus 10 may include a sensor 312 worn on a wrist of a subject to detect a biosignal through the wrist. In addition, the biosignal processing apparatus 10 may be embedded with a processor that processes the biosignal. The embedded processor may generate a biosignal waveform from the biosignal received from the sensor 312, and provide information on a biological condition of a subject (for example, blood pressure information, blood vessel information, or the like) by using the biosignal waveform.

Referring to FIG. 1B, the subject may be provided with information on the biological condition, which is generated by the processor, through a screen displayed on a display 330 of the biosignal processing apparatus 10 worn on the wrist of the subject. Examples of the information on the blood pressure may include numerical information on a minimum blood pressure and a maximum blood pressure of the subject, numerical information on a systolic blood pressure (SBP) and a diastolic blood pressure (DBP) of the subject, information regarding whether a current blood pressure state is normal, and vascular compliance information.

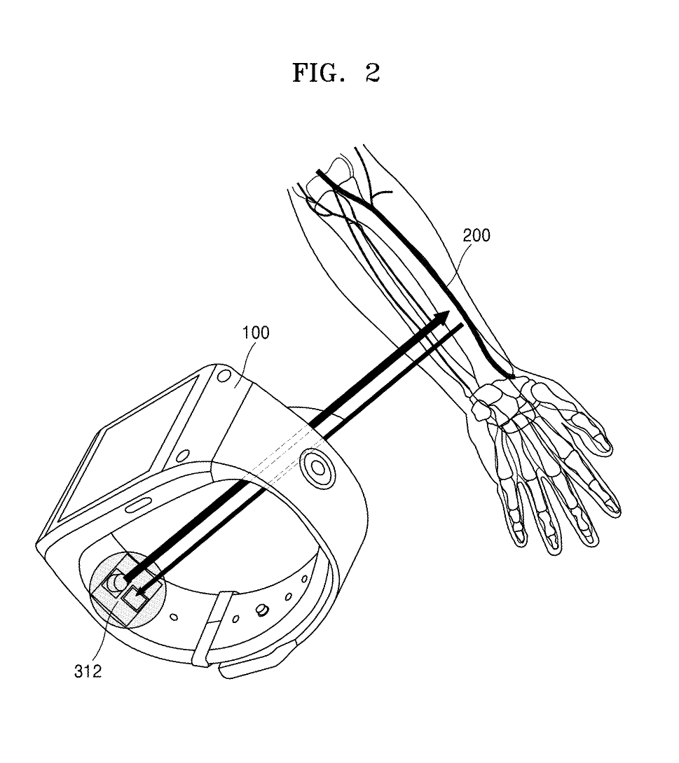

FIG. 2 is a diagram describing an area of a wrist from which the wristwatch type or wristband type biosignal processing apparatus 10 detects a biosignal, according to an exemplary embodiment. Referring to FIG. 2, the biosignal processing apparatus 10 may detect the biosignal by radiating a light beam onto a skin surface close to a radial artery 200 in a contact or non-contact manner. The biosignal may be photoplethysmograph (PPG).

For example, the biosignal processing apparatus 10 may detect a PPG by radiating a light beam onto the radial artery 200 so as to measure an arterial blood pressure. When the PPG is measured on the skin surface of the wrist, underneath which the radial artery 200 passes, a measurement error caused by external factors, such as a thickness of a skin tissue between the skin surface of the wrist and the radial artery 200 may be greatly reduced. In addition, it is known that the radial artery 200 is a blood vessel at which the PPG signal may be detected more accurately than other types of blood vessels inside the wrist.

Therefore, the sensor 312 embedded in the biosignal processing apparatus 10 may be disposed at a position where the sensor 312 is able to detect a light reflected off the skin surface while the subject is wearing the biosignal processing apparatus 10. The biosignal processing apparatus 10 is not limited thereto and may also detect the PPG signal by using blood vessels located at other areas of the wrist, except for the radial artery 200. In FIG. 2, a method of detecting the biosignal by photoelectric conversion has been described. However, the exemplary embodiment is not limited thereto. The biosignal may also be detected by piezoelectric conversion, a mechanical method, or a magnetic method.

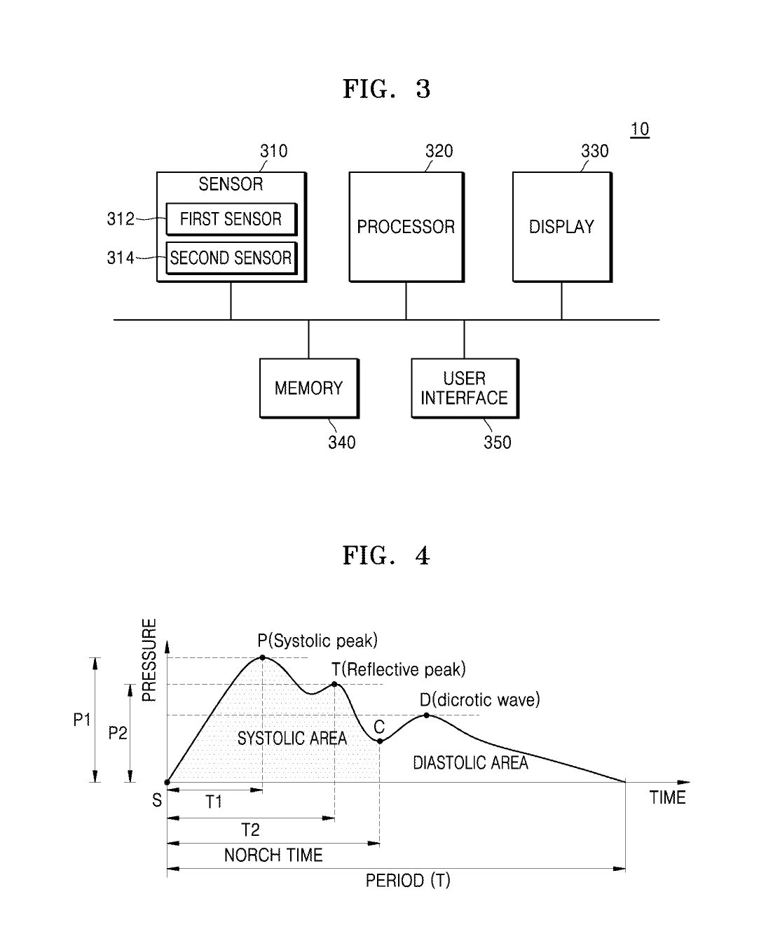

FIG. 3 is a block diagram of the biosignal processing apparatus 10 according to the exemplary embodiment. Referring to FIG. 3, the biosignal processing apparatus 10 may include a sensor 310 that detects a biosignal of a subject and at least one position of a spot (hereinafter, referred to as a detection spot) at which the biosignal is detected, a processor 320 that processes the biosignal by using at least one f the biosignal and the position received from the sensor 310, and estimates information on a biological condition of the subject, a display 330 that displays the information on the biological condition of the subject, a memory 340 that stores data, and a user interface 350 that receives a user input.

The biosignal processing apparatus 10 may be carried by the subject. For example, the biosignal processing apparatus 10 may be a wearable device. For example, the biosignal processing apparatus 10 may be worn on a user's wrist, chest, or ankle. However, the exemplary embodiment is not limited thereto. For example, the sensor 310 may be implemented using a first device capable of being worn on a subject's wrist, and the processor 320, the display 330, the memory 340, and the user interface 350 may be separately implemented using a second device (for example, a mobile terminal).

The sensor 310 may include a first sensor 312 that detects the biosignal of the subject, and a second sensor 314 that detects the position of the detection spot, that is, a position of a spot at which the first sensor 312 is disposed. The first sensor 312 and the second sensor 314 may be implemented using a single device. Therefore, the second sensor 314 may easily detect the position of the first sensor 312.

The first sensor 312 is a sensor that detects the biosignal of the subject, such as an ECG, a galvanic skin reflex (GSR), a PPG, and a pulse wave. The first sensor 312 may detect the biosignal by using a signal reflected after the light beam is irradiated on the subject. However, the exemplary embodiment is not limited thereto. The first sensor 312 may detect the biosignal by applying an electrical signal, a magnetic signal, or a pressure to the subject.

The second sensor 314 is a sensor that detects the position of the spot (detection spot) at which the biosignal is detected, that is, the position of the first sensor 312. The second sensor 314 may be a direction sensor, such as an acceleration sensor, a gyro sensor, a terrestrial magnetic sensor, or a horizontal sensor.

In addition, the second sensor 314 may be used to detect a movement of the subject. When the position of the detection spot is changed within a predetermined time, for example, 10 seconds, the biosignal processing apparatus 10 may determine that the subject moves.

The processor 320 may generate a biosignal waveform from the biosignal. The biosignal waveform may be a time-based function. In addition, the processor 320 may correct the biosignal waveform by using the position received from the second sensor 314. For example, the biosignal may be a PPG according to a heart movement.

Generally, the object that generates the biosignal, for example, the heart, may be disposed in the central area of the subject. The position from which the biosignal is detected, that is, the position of the first sensor 312, for example, a wrist or an ankle, may be an area spaced apart from the heart. Since the object that generates the biosignal and the detection spot of the biosignal are spaced apart from each other, the biosignal may be differently detected according to a relative position change between the object and the detection spot. Since such a position change acts as noise in the biosignal, it is necessary to detect the biosignal at a fixed position. Alternatively, it is necessary to change the biosignal to a biosignal of a fixed position.

The processor 320 may convert the biosignal waveform to a biosignal waveform of a reference position (hereinafter, referred to as a reference biosignal waveform) by using the position of the detection spot. The processor 320 may estimate information on the biological condition of the subject, for example, information on a blood pressure or a vascular compliance, from the reference biosignal waveform.

The processor 320 may be hardware that controls the overall function and operation of the biosignal processing apparatus 10. The processor 320 may be implemented using a single microprocessor module, or may be implemented in a combination of two or more microprocessor modules. That is, the processor 320 is not limited to the above-described implementation forms.

The display 330 may display the information on the biological condition of the subject, which is estimated by the processor 320. For example, the display 330 may include an output module, such as a display panel, a liquid crystal display (LCD) screen, or a light-emitting display (LED) screen, which is provided in the biosignal processing apparatus 10. However, according to the exemplary embodiment the display 330 may be omitted from the biosignal processing apparatus 10 and may output the biosignal processed by the processor 320 to an external display device.

The memory 340 may store data necessary for operations of the biosignal processing apparatus 10. According to an exemplary embodiment, the memory 340 may be a general storage medium, such as a hard disk drive (HDD), a read only memory (ROM), a random access memory (RAM), a flash memory, and a memory card.

The memory 340 may store a transfer function for converting the biosignal waveform to the reference biosignal waveform. The memory 340 may store the transfer function as metadata defined at each position. Therefore, the processor 320 may read the transfer function corresponding to the position of the detection spot from the memory 340 and convert the biosignal waveform to the reference biosignal waveform by using the read transfer function.

The user interface 350 may receive an input for operating the biosignal processing apparatus 10 from the subject, and may output the information on the biological condition processed by the processor 320. The user interface 350 may include a button, a keypad, a switch, a dial, or a touch interface, which allows the subject to directly operate the biosignal processing apparatus 10. The user interface 350 may include a display 330 that displays an image and may be implemented using a touch screen. According to another exemplary embodiment, the user interface 350 may include an input/output (I/O) port that connects human interface devices (HIDs). The user interface 350 may include an I/O port that inputs or outputs an image.

FIG. 4 is a graph of the biosignal waveform according to the exemplary embodiment. In FIG. 4, a PPG is illustrated as the biosignal waveform. The biosignal waveform may include a plurality of factors capable of defining a relationship between a blood and a blood vessel according to a heart movement. Specifically, before a formation of a waveform, a left ventricle contracts and a pressure of the left ventricle increases. Thus, an aortic valve is opened. At this time, a spot at which a blood of the left ventricle starts escaping from an aortic arch may be defined as a first factor S. Then, a blood flows from the left ventricle to the aortic arch at a fast speed. At this time, a spot at which a pressure and a volume of a blood vessel reaches a peak may be defined as a second factor P. The pressure of the second factor P may indicate an ability to discharge the blood of the left ventricle and the vascular compliance of the aorta.

When the escape amount of the blood is reduced, the pressure and the volume are reduced. At a certain position, the reducing speed becomes slow for a moment. This spot may be defined as a third factor T. The generation cause of the third factor T affects the pressure and the volume because a component of a previously generated wave is reflected again and returned from a peripheral branch. The pressure and the time of the third factor T may be used to define the compliance of the blood vessel.

The fourth factor C is a spot at which the pressure of the left ventricle is sufficiently lower than the pressure for escaping the blood to the aortic arch. The fourth factor C is a spot at which a mesenteric aorta is closed and a spot at which a right atrium contracts and a left ventricle relaxes. The pressure of the fourth factor C is associated with afterload. When a peripheral resistance of a blood vessel increases, the pressure of the fourth factor C also increases. A spot at which the pressure and the volume of the artery slightly increases after the aortic valve is closed may be the fifth factor D. When a difference between the fifth factor (D point) and the fourth factor (C point) is reduced or is close to zero, it may indicate that the aortic valve opening/closing function is abnormal.

As described above, time of the factors, time interval between the factors, pressure or pressure difference of the factors, and the like may be factors that determine information on the biological condition, such as the blood pressure of the subject, the vascular compliance, and normality or abnormality of the aortic valve or venous valve opening/closing function.

On the other hand, the movement of the subject may change the relative position between the heart and the detection spot, that is, the relative position between the heart and the spot at which the first sensor 312 is disposed, for example, positions of the heart and the wrist. A height difference between the heart and the detection spot (for example, the wrist) may generate a change in the blood pressure according to the gravity. In addition, an arrival time of a reflect wave may be changed according to the gravity. Hence, the biosignal waveform may be changed according to the relative position between the heart and the detection spot.

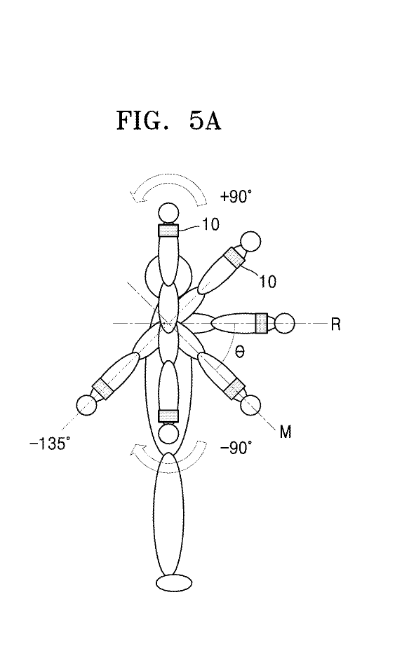

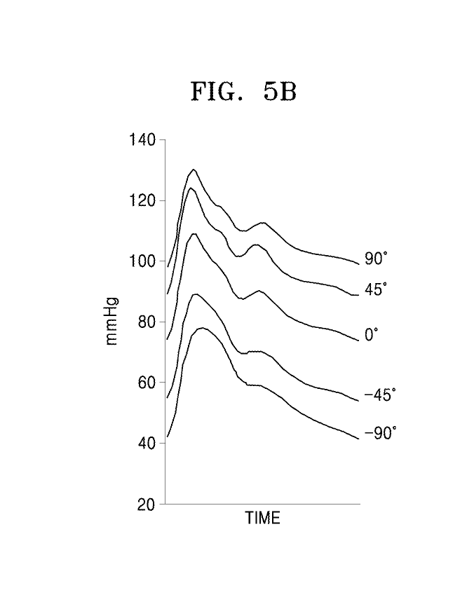

FIG. 5A is a diagram illustrating a change in positions of detection spots with respect to a heart, and FIG. 5B is a graph of biosignal waveforms measured at the respective detection spots of FIG. 5A. The measured biosignal waveform may be a PPG waveform. For convenience, it is assumed that the detection spot is the wrist of the subject. That is, the first sensor 312 may be worn on the wrist of the subject.

A position of a detection spot when the heart and the detection spot are at the same height is referred to as a reference position. The subject may rotate his or her arm in a clockwise or counterclockwise direction at the reference position. When the subject rotates his or her arm in a clockwise direction at the reference position, the position of the first sensor 312 becomes lower than the reference position. When the subject rotates his or her arm in a counterclockwise direction at the reference position, the position of the first sensor 312 becomes higher than the reference position.

When a distance between the heart and the reference position is a reference line R and a distance between the heart and the first sensor 312 is a measurement line M, an angle between the reference line R and the measurement line M is referred to as an in-between angle θ. When the arm is rotated in a clockwise direction, the in-between angle θ becomes a negative value, and when the arm is rotated in a counterclockwise direction, the in-between angle θ becomes a positive value.

As illustrated in FIGS. 5A and 5B, when the in-between angle θ is −90 degrees, −45 degrees, 0 degree, 45 degrees, and 90 degrees, the biosignal waveform may be changed according to the in-between angle θ. According to the biosignal waveform, a magnitude of the biosignal waveform when the in-between angle θ is positive with respect to the same spot is greater than a magnitude of the reference biosignal waveform. A magnitude of the biosignal waveform when the in-between angle θ is negative with respect to the same spot is smaller than a magnitude of the reference biosignal waveform. This is due to the influence of the gravity according to the height difference between the heart and the detection spot.

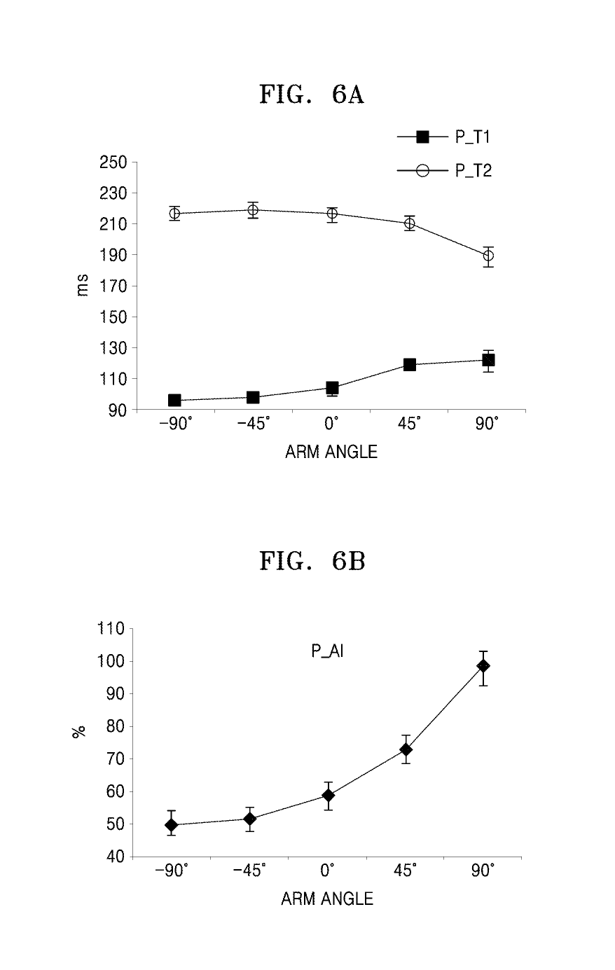

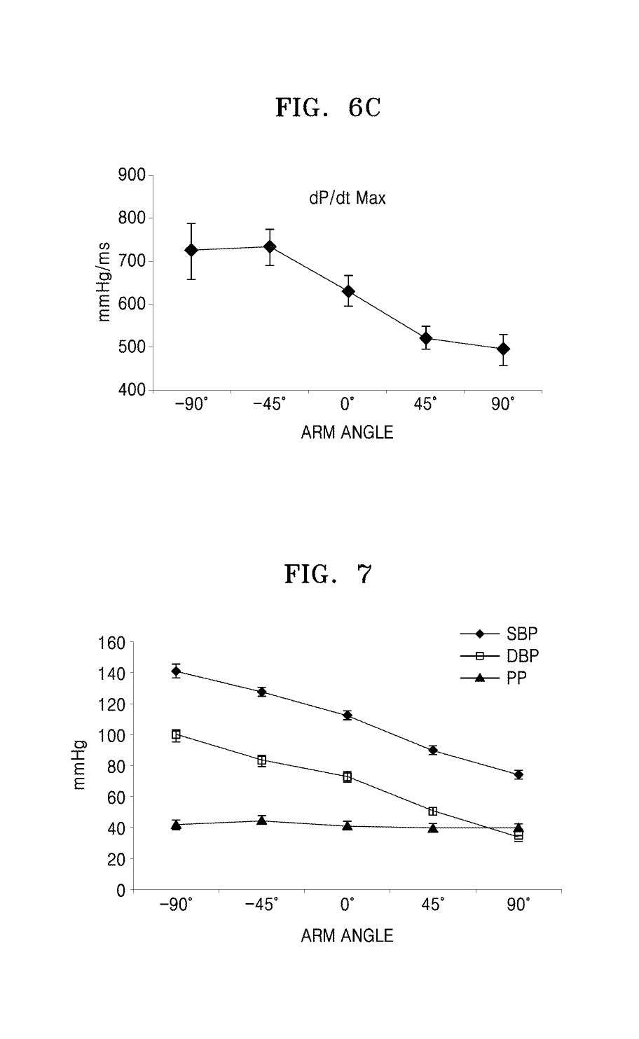

In addition to the magnitude of the biosignal waveform, the time, the time interval, and the magnitude of the factors of the biosignal waveform may also be changed according to the position of the detection spot. FIG. 6A is a graph of a maximum systolic time and a reflect wave time according to a position of a detection spot. The maximum systolic time is a time interval T1 between the first factor S and the second factor P in FIG. 4, and the reflect wave time is a time interval T2 between the first factor S and the third factor T in FIG. 4. It can be seen that the maximum systolic time and the reflect wave time are changed according to the position of the detection spot.

FIG. 6B is a graph of an augmentation index (AI) according to the position of the detection spot. The augmentation index is the product of the magnitude P1 of the magnitude P2 of the third factor T and 100 with respect to the magnitude P1 of the second factor P. It can be seen that the augmentation index is changed according to the position of the detection spot.

FIG. 6C is a graph of a peak systolic velocity according to the position of the detection spot. The systolic velocity indicates a magnitude between the first factor S and the second factor P with respect to the time interval T1 between the first factor S and the second factor P. It can be seen that the systolic velocity is changed according to the position of the detection spot. As illustrated in FIGS. 6A to 6C, it can be seen that the factors of the biosignal waveform also are changed according to the position of the detection spot.

Since the biosignal waveform is changed according to the position of the detection spot, the information on the biological condition, which is estimated from the biosignal waveform, may also be changed. FIG. 7 is a graph of a blood pressure according to a position of a detection spot. The systolic blood pressure is a blood pressure when a heart contracts and a blood is pushed out toward an artery, and the diastolic blood pressure is a blood pressure when a ventricle expands and a blood is not pushed out. In addition, the pulse pressure is a difference between the systolic blood pressure and the diastolic blood pressure. Even though the pulse pressure is not changed according to the position of the detection spot, the systolic blood pressure and the diastolic blood pressure are changed according to the position of the detection spot.

As such, since the biosignal waveform is changed according to the position of the detection spot, it is necessary to generate the biosignal waveform having no relation to the position of the detection spot. The biosignal processing apparatus 10 according to the exemplary embodiment may include the second sensor 314 that detects the detection spot, that is, the position of the first sensor 312. When the subject maintains his or her arm in an unfolded state, the second sensor 314 may be a direction sensor that detects a relative position between the heart and the arm as a direction. For example, the second sensor 314 may be a 1-axis horizontal sensor. For example, the second sensor 314 may be a tilt sensor, an output voltage of which is changed according to a tilt value of the sensor. The subject may wear the biosignal processing apparatus 10 such that the axis of the horizontal sensor is disposed in parallel to the axis of the arm. Therefore, the relative position of the detection spot may be determined based on the tilt value measured by the tilt sensor.

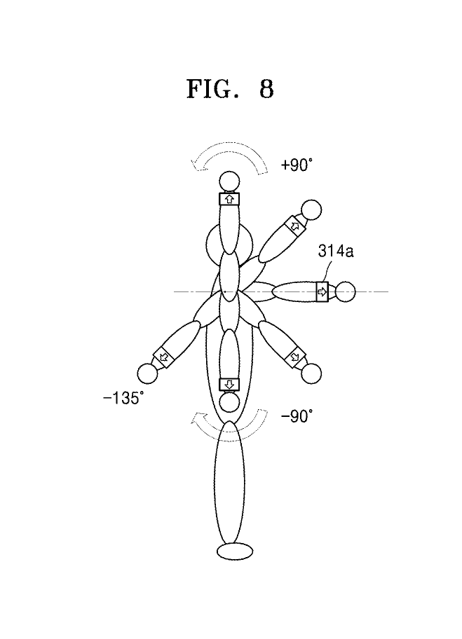

FIG. 8 is a diagram illustrating a direction of a sensor 314a with respect to a position of a detection spot when a 1-axis horizontal sensor 314a is worn, according to an exemplary embodiment. As illustrated in FIG. 8, when the axis of the horizontal sensor 314a worn on the wrist of the subject indicates the hand of the subject, one axial direction of the horizontal sensor 314a is changed one to one according to the position of the detection spot.

For example, when the horizontal sensor 314a is the tilt sensor and 0 degree of the tilt sensor is set to be matched with the reference line, the tilt sensor may measure the tilt value and detect an angle between the measurement line and the reference line based on the measured tilt value. That is, the tilt value of the tilt sensor may be an angle between the measurement line and the reference line. Therefore, the relative position between the heart and the detection spot may be determined by using the result of the second sensor, and the biosignal waveform may be converted to the reference biosignal waveform by using the relative position and the transfer function. The transfer function will be described below.

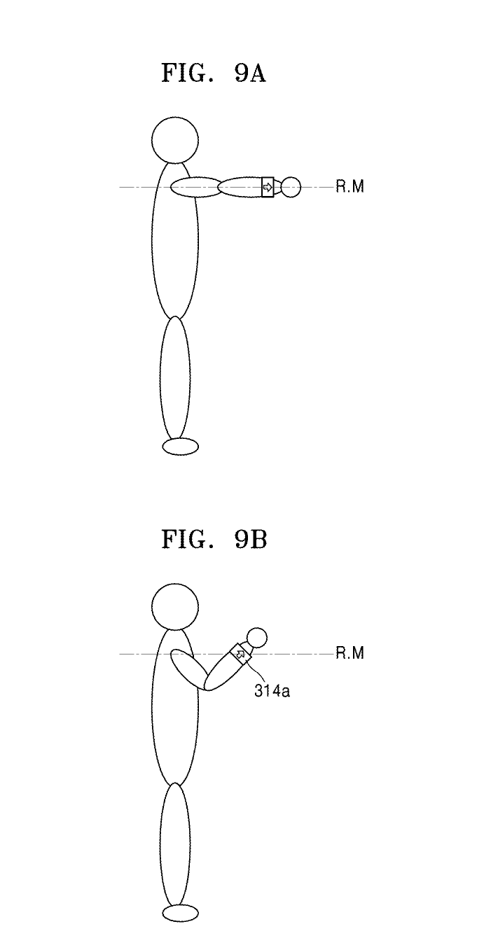

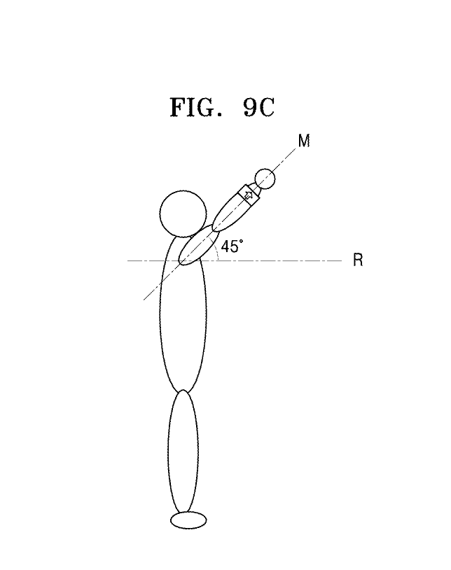

On the other hand, the subject may fold his or her arm. The relative position of the detection spot may not correspond to the detection result of the horizontal sensor 314a one to one. FIG. 9A to 9C are reference diagrams describing the relative position of the detection spot and the detection result of the horizontal sensor, according to an exemplary embodiment. As illustrated in FIG. 9A, when the subject unfolds his or her arm such that the angle between the reference line R and the measurement line M becomes 0 degree, the detection result of the horizontal sensor 314a may be 0 degree. As illustrated in FIG. 9B, the subject may fold his or her arm such that the angle between the reference line R and the measurement line M becomes 0 degree. At this time, the detection result of the horizontal sensor 314a may be +45 degrees. In addition, as illustrated in FIG. 9C, the subject may unfold his or her arm such that the angle between the reference line R and the measurement line M becomes +45 degrees. At this time, the detection result of the horizontal sensor 314a may be +45 degrees.

As illustrated in FIGS. 9A and 9B, even when the relative positions of the detection spot are equal to each other, the detection results of the horizontal sensor 314a may be different from each other. In addition, as illustrated in FIGS. 9B and 9C, even when the relative positions of the detection spot are different from each other, the detection results of the horizontal sensor 314a may be equal to each other.

In such cases, the position of the detection spot calculated from the detection result of the horizontal sensor 314a without considering other control factors may have a low accuracy. Therefore, the biosignal processing apparatus according to the exemplary embodiment may determine the position of the detection spot by using the biosignal waveform.

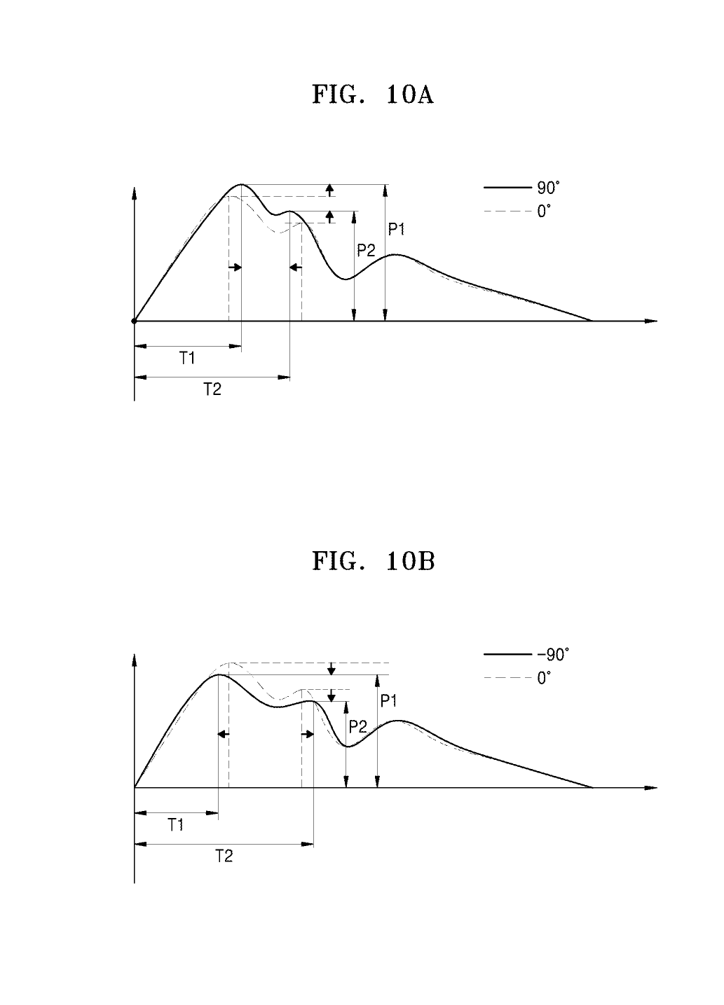

FIGS. 10A and 10B are diagrams illustrating a change in the biosignal waveform with respect to the detection spot, according to an exemplary embodiment. FIG. 10A illustrates a comparison between the biosignal waveform when the angle of the detection spot is 0 degree and the biosignal waveform when the angle of the detection spot is +90 degrees. It can be seen that even though the period of the +90-degree biosignal waveform is equal to the period of the 0-degree biosignal waveform, the augmentation index (AI) of the +90-degree biosignal waveform increases, the maximum systolic time T1 is lengthened, and the reflect wave time T2 is shortened.

FIG. 10B illustrates a comparison between the biosignal waveform when the angle of the detection spot is 0 degree and the biosignal waveform when the angle of the detection spot is −90 degrees. It can be seen that even though the period of the −90-degree biosignal waveform is equal to the period of the 0-degree biosignal waveform, the augmentation index (AI) of the −90-degree biosignal waveform increases, the maximum systolic time T1 is shortened, and the reflect wave time T2 is lengthened. This is because the biosignal waveform is affected by the gravity.

However, when there occurs a physiological change, such as a subject's drug ingestion or movement, the period or the like of the biosignal waveform is also changed in a different form from the change in the factors according to the change in the position of the detection spot as described above. For example, the period may be changed, and the maximum systolic time T1 and the reflect wave time T2 may be shortened or lengthened at the same time.

Therefore, when the augmentation index (AI) increases while the period of the biosignal waveform is equal, the maximum systolic time T1 is lengthened but the reflect wave time T2 is shortened, the biosignal processing apparatus may determine that the detection spot is changed to over the reference line. When the augmentation index (AI) decreases while the period of the biosignal waveform is equal, the maximum systolic time T1 is shortened but the reflect wave time T2 is lengthened, the biosignal processing apparatus may determine that the detection spot is changed to below the reference line. The degree of change of the detection spot may be more accurately determined by the change amounts of the augmentation index P, the maximum systolic time T1, and the reflect wave time T2.

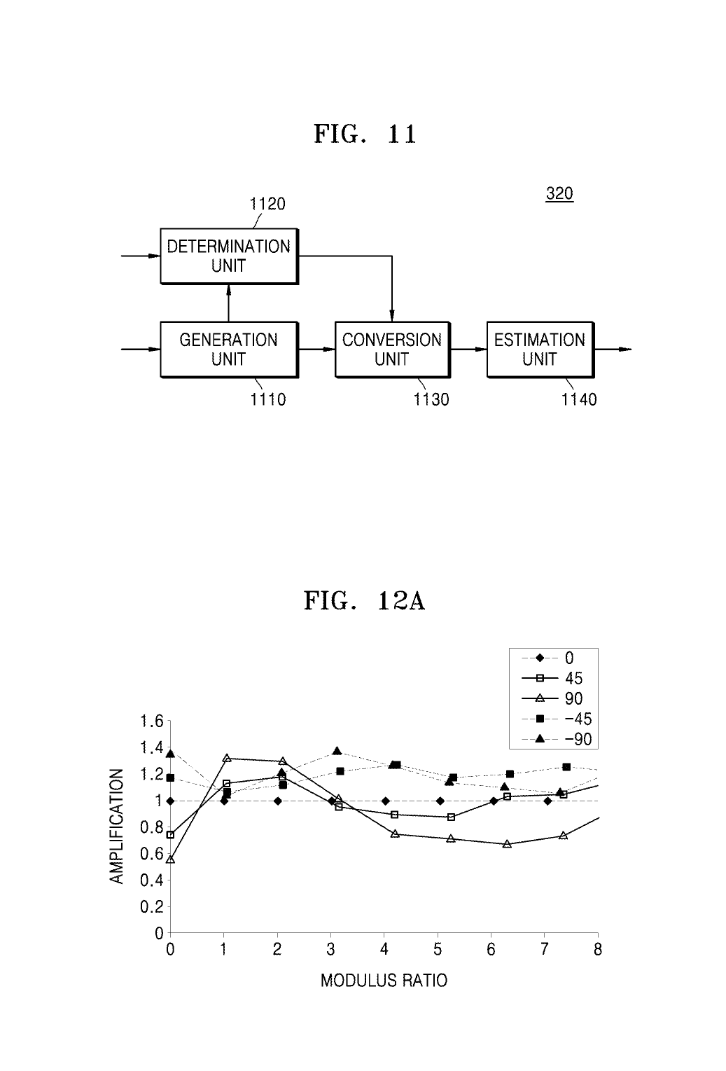

FIG. 11 is a block diagram of the processor 320 of FIG. 3. Referring to FIG. 11, the processor 320 may include a generation unit 1110 that generates the biosignal waveform by using the biosignal received from the first sensor 312, a determination unit 1120 that determines the position of the detection spot, that is, the position of the first sensor 312, a conversion unit 1130 that converts the biosignal waveform to the reference biosignal waveform, and an estimation unit 1140 that estimates the information on the biological condition of the subject from the reference biosignal waveform.

The generation unit 1110 may receive the biosignal from the first sensor 312 and generate the biosignal waveform according to time. When generating the biosignal waveform, the generation unit 1110 may amplify the received biosignal, for example, the PPG, and filter the amplified PPG by using a FIR bandpass filter. The factors may be detected from the filtered PPG and the biosignal waveform may be generated by adaptively filtering the detected factors. Since the biosignal, in particular the biosignal from the heart, may have a periodic waveform, the biosignal waveform may be a waveform in which a periodic signal is repeated.

The determination unit 1120 may determine the relative position of the detection spot at which the biosignal is detected with respect to the position of the heart that generates the biosignal. For example, when the second sensor 314 is a direction sensor, the determination unit 1120 may determine the relative position of the detection spot by using the detected direction. Referring to FIG. 7, when the result received from the second sensor 314 is −90 degrees, the determination unit 1120 may determine that the relative position of the detection spot is −90 degrees from the reference line.

In addition, the determination unit 1120 may determine the relative position of the detection spot by using the biosignal waveform. For example, the memory 340 may store information on the change amounts of the factors of the biosignal with respect to the degree of change of the respective positions. The determination unit 1120 may determine the relative position of the detection spot by extracting at least two factors from the biosignal waveform and extracting the position information corresponding to the factor values. As illustrated in FIGS. 6A to 6C, different factor values of the biosignal waveform according to the relative position of the detection spot are used.

Alternatively, the determination unit 1120 may determine the relative position of the detection spot by using the result received from the second sensor 314 and the change in the biosignal waveform. Even when the detection result of the second sensor 314 is changed, if the period, the augmentation index (AI), the maximum systolic time T1, and the reflect wave time T2 are equal, the determination unit 1120 may determine that the position of the detection spot is not changed. However, even when the detection result of the second sensor 314 is not changed, if the augmentation index (AI), the maximum systolic time T1, and the reflect wave time T2 are changed while the period of the biosignal waveform is equal, the determination unit 1120 may determine the relative position of the detection spot based on the change rate of the augmentation index (AI), the maximum systolic time T1, and the reflect wave time T2.

The conversion unit 1130 may convert the biosignal waveform generated by the generation unit 1110 to the reference biosignal waveform by using the position determined by the determination unit 1120. The transfer function for converting the biosignal waveform may be used. The transfer function is a function that defines the relationship for converting the biosignal waveform of the detection spot to the reference biosignal waveform. The transfer function may be prestored in the memory 340 as metadata for each position.

The transfer function may be modeled for each individual, or may be generalized regardless of an individual. Alternatively, the generalized transfer function stored in the memory 340 may be modified according to individuals when each individual uses the biosignal processing apparatus 10.

Since the biosignal processing apparatus 10 according to the exemplary embodiment merely uses the transfer function and does not calculate the transfer function, the method of modeling the transfer function will be described briefly below. The method of modeling the transfer function may be executed according to the biosignal processing apparatus 10. Alternatively, the method of modeling the transfer function may be executed by an external device, and the execution result may be stored in the biosignal processing apparatus 10. Thus, a device for modeling the transfer function is also referred to as a modeling device. First, the biosignal waveform for each detection spot may also be stored in the modeling device.

For example, the biosignal waveforms corresponding to −90 degrees, −45 degrees, 0 degree, 45 degrees, and 90 degrees may be stored in the modeling device. The modeling device may calculate the transfer function between the biosignal waveforms corresponding to −90 degrees, −45 degrees, 45 degrees, and 90 degrees and the biosignal waveform corresponding to 0 degree. For convenience, the biosignal waveforms corresponding to nonzero angles (a), such as −90 degrees, −45 degrees, 45 degrees, and 90 degrees, are referred to as candidate waveforms, and the biosignal waveform corresponding to 0 degree (hereinafter, referred to as a target angle) is referred to as a target waveform.

The modeling device may perform discrete Fourier transform on the candidate waveform for each frequency so as to divide the candidate waveform into an amplitude part (Ma(f)) and a phase part (Pa(f)). Here, f is an operating frequency of the device that generates the biosignal waveform. In addition, the modeling device may also perform discrete Fourier transform on the target waveform for each frequency so as to divide the target waveform into an amplitude part (MO(f)) and a phase part (PO(f)).

The modeling device may define the transfer function by defining a first transfer function of the amplitude part as an amplitude ratio nd defining a second transfer function of the phase part as a phase difference. For example, the modeling device may define the first transfer function (TMa) as the amplitude ratio of the amplitude part of the discrete-Fourier-transformed candidate waveform with respect to the amplitude part (MO) of the discrete-Fourier-transformed target waveform, and define the second transfer function (TPa) as the phase difference of the phase part (Pa) of the discrete-Fourier-transformed candidate waveform with respect to the phase part (PO) of the discrete-Fourier-transformed target waveform, as expressed in Equation 1 below.

TMa(f)=Ma(f)/M0(f)

TPa(f)=Pa(f)−P0(f) [Equation 1]

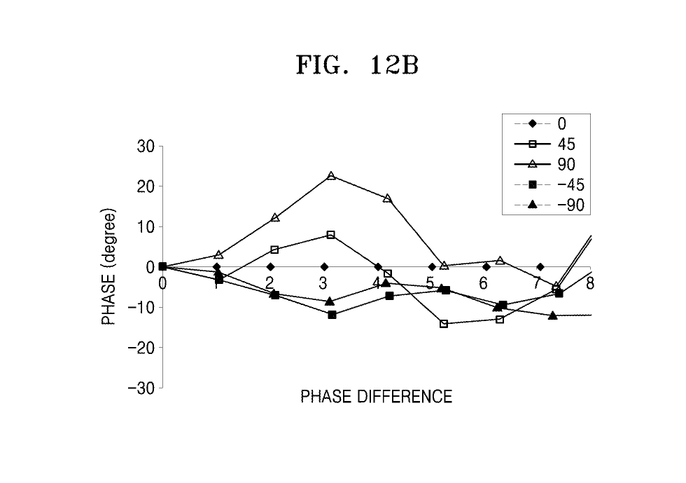

FIG. 12A is a graph of the first transfer function for the detection spot, and FIG. 12B is a graph of the second transfer function for the detection spot. As illustrated in FIGS. 12A and 12B, the first transfer function may be calculated according to the relative position of the detection spot for each frequency and the second transfer function according to the relative position of the detection spot for each frequency.

Therefore, the conversion unit 1130 may convert the biosignal waveform to the reference biosignal waveform (for example, the biosignal waveform when the detection spot is 0 degree) by using the relative position of the detection spot and the transfer function. Specifically, the conversion unit 1130 may divide a biosignal waveform, which is applied by the generation unit 1110, into an amplitude part (Mθ(f)) and a phase part (Pθ(f)) by performing discrete Fourier transform thereon.

The conversion unit 1130 may read, from the memory 340, transfer functions, that is, the first transfer function and the second transfer function, corresponding to the relative position of the detection spot determined by the determination unit 1120. Then, the conversion unit 1130 may acquire a converted amplitude part (M′0(f)) and a converted phase part (P′0(f)) by applying the first transfer function (TMa) to the amplitude part (Mθ) and applying the second transfer function (TPa) to the phase part (Pθ), as expressed in Equation 2 below.

TMa(f)=Ma(f)/M0(f)

M′0(f)=M0(f)/TMθ(f)

P′0(f)=P0(f)−TPθ(f) [Equation 2]

TMθ(f) is the first transfer function when the candidate angle is θ, and TPθ(t) is the second transfer function when the candidate angle is θ.

The reference biosignal waveform may be acquired by performing an inverse discrete Fourier transform on the amplitude part (M′0(f)) and the phase part (P′0(f)) to which the transfer function is applied. The reference biosignal waveform is a biosignal waveform at a reference position, and the reference position may be a position when the first sensor 312 and the heart are located at the same height. However, the exemplary embodiment is not limited thereto. The reference position may be a position when the detection spot is located below the heart, and may be changed by a designer.

The estimation unit 1140 may estimate the information on the biological condition of the subject by using the reference biosignal waveform. For example, when the biosignal waveform is a PPG waveform, the estimation unit 1140 may estimate the information on the biological condition, such as the systolic blood pressure, the diastolic blood pressure, and the vascular compliance, by using the PPG waveform and display the estimation result on the display 330.

When a pressure is estimated from the PPG waveform, a blood pressure estimation model may be applied. The blood pressure estimation model may be a linear model or a non-linear model. The non-linear model may include a neural network learning model, a model for comparison with a blood pressure measured by a cuff blood pressure monitor, and the like.

For example, the estimation unit 1140 may apply factors extracted from the PPG waveform to the neural network learning model. More specifically, the neural network learning model for the blood pressure estimation is a model that, when specific factors are input as query, outputs a final blood pressure matched with the input factors by using a previously learnt neural network data set. The neural network data set may correspond to a type of database that is previously learnt through data mining with respect to the correlation of the factors in the PPG waveform and the blood pressure. Therefore, the estimation unit 1140 may acquire the final blood pressure from the previously learnt neural network data set.

On the other hand, as described above, since it is apparent to those skilled in the art that the factors extracted from the PPG waveform are used for estimating the blood pressure in the neural network learning model or the linear mode, a detailed description thereof will be omitted. In addition, since various linear models or non-linear models for estimating the blood pressure are known and obvious to those skilled in the art, a detailed description thereof will be omitted. Furthermore, besides the blood pressure, the estimation unit 1140 may estimate diseases, such as autonomic nervous system (ANS) abnormality and stress degrees, by using the PPG waveform.

The processor 320 may determine whether the information on the biological condition, which is generated by the estimation unit 1140, is in a normal range or an abnormal range, and display the determination result on the display 330. When the information on the biological condition is in the abnormal range, the processor 320 may provide a subject action guide such that the information on the biological condition falls within the normal range.

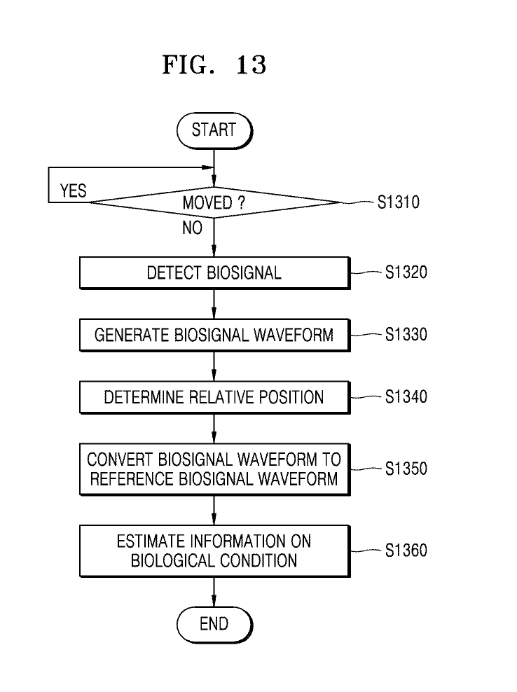

FIG. 13 is a flowchart of a biosignal processing method according to an exemplary embodiment.

In operation 1310, the processor 320 may determine whether a subject moves. For example, when a position of a detection spot, which is received from the second sensor 314, is changed, the processor 320 may determine that the subject moves. Since the movement of the subject may act as noise in a biosignal, the biosignal processing apparatus 10 may detect a biosignal when there is no movement of the subject. However, the exemplary embodiment is not limited thereto. The biosignal may be detected even in a moving state by removing the effect of the subject's movement from the biosignal.

In operation 1320, the first sensor 312 may detect the biosignal of the subject. The biosignal may be generated by a movement of a heart. The first sensor 312 may detect the biosignal from a part of the subject in a non-invasive manner. For example, the first sensor 312 may be disposed on a wrist of the subject to detect the biosignal of the subject by using a light beam.

In operation 1330, the generation unit 1110 of the processor 320 may generate a biosignal waveform according to time by using the biosignal received from the first sensor 312. When the biosignal waveform is generated, a noise removal filter may be used.

In operation 1340, the determination unit 1120 of the processor 320 may determine a relative position of a detection spot with respect to the heart. The determination unit 1120 may determine the relative position of the detection spot by using at least one selected from the detection result of the second sensor 314 and the biosignal waveform. The method of determining the relative position will be described below.

In operation 1350, the conversion unit 1130 may convert the biosignal waveform to a reference biosignal waveform by using the relative position of the detection spot. For example, the memory 340 may prestore metadata in which the transfer function for converting the biosignal waveform to the reference biosignal waveform is defined for each position. The conversion unit 1130 may read the transfer function corresponding to the relative position of the detection spot from the metadata and convert the biosignal waveform to the reference biosignal waveform by using the read transfer function. The transfer function may be divided into a first transfer function defined as an amplitude ratio and a second transfer function defined as a phase difference. Specifically, the biosignal waveform may be divided into an amplitude part and a phase part by using a discrete Fourier transform. The first transfer function may be applied to the amplitude part, and the second transfer function may be applied to the phase part. Then, the reference biosignal waveform may be acquired by using an inverse discrete Fourier transform.

In operation 1360, the estimation unit 1140 may estimate the information on the biological condition of the subject by analyzing the biosignal waveform of the reference spot. The information on the biological condition of the subject may be blood pressure information, vascular compliance information, and the like.

As described above, the relative position of the detection spot may be determined by using at least one selected from the detection result of the second sensor 314 and the biosignal waveform. When the detection result of the second sensor 314 corresponds to the relative position of the detection spot one to one, the determination unit 1120 may determine the relative position of the detection spot from the detection result of the second sensor 314.

For example, when the biosignal processing apparatus 10 generates the biosignal waveform, the biosignal processing apparatus 10 may display or readout guidelines including information that the subject is advised to maintain his or her arm in an unfolded state. When the subject's arm is being unfolded, the detection result of the second sensor 314 and the relative position of the detection spot may correspond to each other one to one as illustrated in FIG. 5A. Alternatively, when the biosignal processing apparatus 10 generates the biosignal waveform, the subject may wear the biosignal processing apparatus 100 on his or her forearm. When the biosignal processing apparatus 10 is worn on the forearm, the detection result of the second sensor 314 and the relative position of the detection spot may correspond to each other one to one. In addition, when the detection result of the second sensor 314 and the relative position of the detection spot do not correspond to each other one to one, that is, when it is expected that the relative position of the detection spot is plural as the detection result of the second sensor 314, the determination unit 1120 may not determine the relative position of the detection spot and the generation unit 1110 may not generate the biosignal waveform.

Alternatively, the determination unit 1120 may determine the relative position of the detection spot based on the biosignal waveform. FIG. 14 is a flowchart of the method of determining the relative position of the detection spot from the biosignal waveform, according to an exemplary embodiment.

In operation 1410, the determination unit 1120 receives the biosignal waveform from the generation unit 1110. In operation 1420, the determination unit 1120 extracts a plurality of factors from the biosignal waveform. The factors may include the period (T), the augmentation index (AI), the maximum systolic time (T1), and the reflect wave time (T2) of the biosignal waveform.

The determination unit 1120 may determine the relative position of the detection spot by comparing the extracted factors with the factors of the reference biosignal waveform. For convenience, the biosignal waveform, of which the relative position of the detection spot is to be determined is referred to as a current biosignal waveform, and the biosignal waveform that is referenced for determining the relative position of the current biosignal waveform is referred to as a reference biosignal waveform. The reference biosignal waveform may be a biosignal waveform, of which the relative position of the detection spot is previously determined and which is generated prior to the current biosignal waveform.

Specifically, in operation 1430, the determination unit 1120 determines whether the period of the current biosignal waveform is equal to the period of the reference biosignal waveform. When the period of the current biosignal waveform is not equal to the period of the reference biosignal waveform, the position of the detection spot is not changed but the biosignal of the subject is changed in itself.

However, in operation 1440, when the period of the current biosignal waveform is equal to the period of the reference biosignal waveform, the determination unit 1120 may determine the relative position of the detection spot by comparing the factors of the current biosignal waveform with the factors of the reference biosignal waveform.

For example, when the augmentation index (AI) of the current biosignal waveform is higher than the augmentation index (AI) of the reference biosignal waveform, the maximum systolic time (T1) is lengthened, but the reflect wave time (T2) is shortened, the determination unit 1120 may determine that the relative position of the detection spot corresponding to the current biosignal waveform becomes higher than the relative position of the detection spot corresponding to the reference biosignal waveform.

In addition, when the augmentation index (AI) of the current biosignal waveform is lower than the augmentation index (AI) of the reference biosignal waveform, the maximum systolic time (T1) is shortened, but the reflect wave time (T2) is lengthened, the determination unit 1120 may determine that the relative position of the detection spot corresponding to the current biosignal waveform becomes lower than the relative position of the detection spot corresponding to the reference biosignal waveform.

The degree of change of the relative position may be determined by the change amounts of the augmentation index (AI), the maximum systolic time (T1), and the reflect wave time (T2). The information on the degree of change of the relative position according to the change amounts of the augmentation index (AI), the maximum systolic time (T1), and the reflect wave time (T2) may be prestored in a metadata format. The determination unit 1120 may determine the change degree of the relative position by using the metadata.

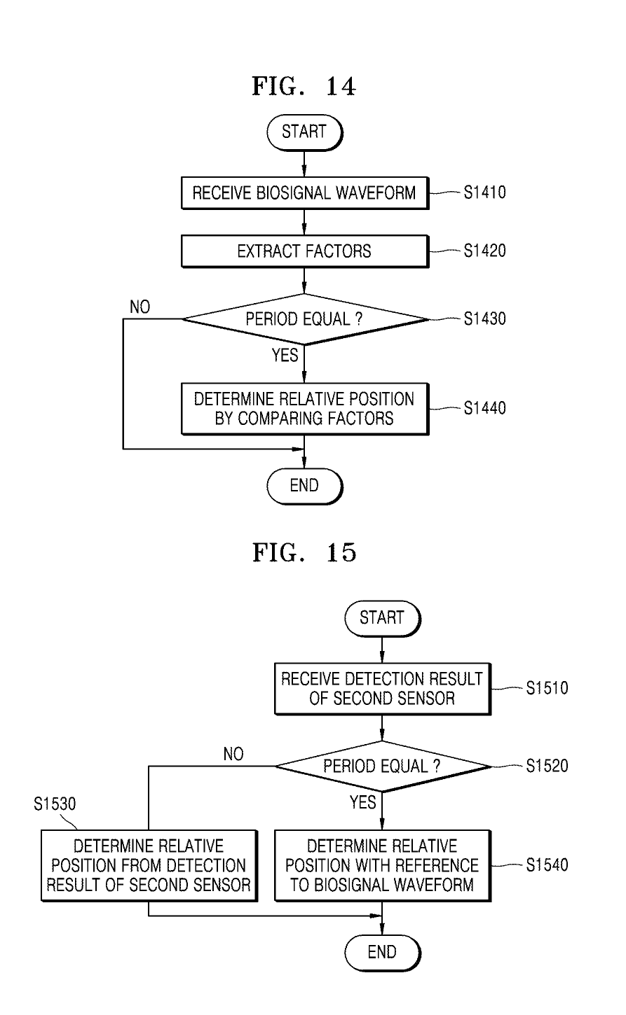

Alternatively, the determination unit 1120 may determine the relative position of the detection spot by using both of the change of the biosignal waveform and the detection result of the second sensor 314. FIG. 15 is a flowchart of a method of determining a relative position of a detection spot, according to another exemplary embodiment.

In operation 1510, the determination unit 1120 receives the detection result of the second sensor 314.

In operation 1520, the determination unit 1120 determines whether a plurality of relative positions are expected from the detection result of the second sensor 314. For example, when the biosignal processing apparatus is worn on the wrist of the subject and the detection result of the second sensor 314 is −180 degrees to −145 degrees, a state in which the arm is unfolded may be maintained. Therefore, when the detection result of the second sensor 314 is −180 degrees to −145 degrees, the determination unit 1120 may expect that the relative position of the detection spot is single. However, when the detection result of the second sensor 314 is −145 degrees to +180 degrees, the relative position of the subject may be changed according to a state in which the arm is unfolded and a state in which the arm is folded. Therefore, when the detection result of the second sensor 314 is −145 degrees to +180 degrees, the determination unit 1120 may expect that the relative position of the detection spot is plural.

In operation 1530, when the single relative position is expected from the detection result of the second sensor 314, the determination unit 1120 may determine the relative position of the detection spot from the detection result of the second sensor 314. That is, the determination unit 1120 may finally determine the expected single relative position as the relative position of the detection spot.

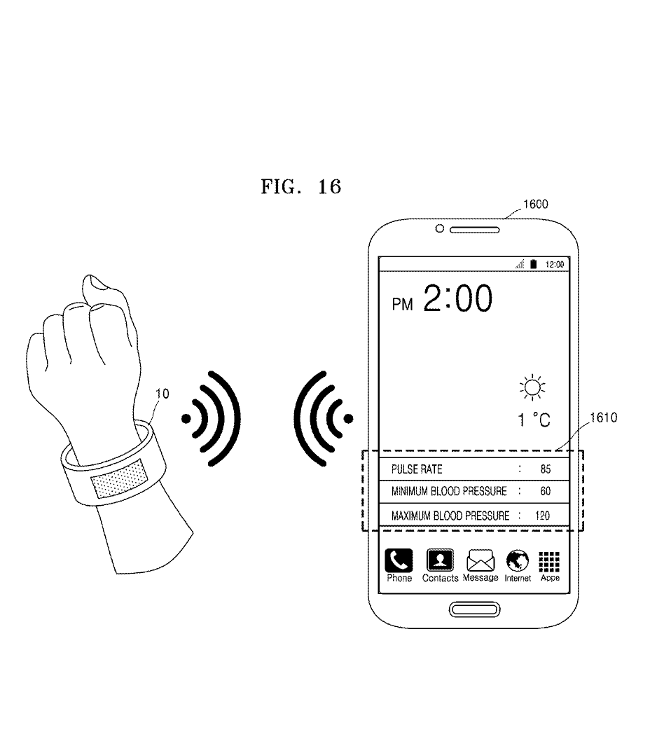

However, in operation 1540, when a plurality of relative positions are expected from the detection result of the second sensor 314, the determination unit 1120 may determine any one of the plurality of expected relative positions as the relative position of the detection spot with reference to the biosignal waveform. Since the method of determining the relative position of the detection spot from the biosignal waveform has been described above with reference to FIG. 14, a detailed description will be described. FIG. 16 is a reference diagram describing a method by which the biosignal processing apparatus 10 provides information on a blood pressure, according to another exemplary embodiment.

Referring to FIG. 16, in a case where the biosignal processing apparatus 10 is provided with a wireless communication function, such as Bluetooth or WiFi, the biosignal processing apparatus 10 may transmit monitored blood pressure information 1610 to a smartphone 1600 of a subject by using the wireless communication function. Therefore, the subject may receive the blood pressure information 1610 through a display screen of the smartphone 1600, in addition to the biosignal processing apparatus 10.

In addition, other exemplary embodiments can also be implemented through computer readable code/instructions stored in/on a non-transitory medium, e.g., a computer readable medium, to control at least one processing element to implement any above described exemplary embodiment. The medium can correspond to any medium/media permitting the storage and/or transmission of the computer readable code.

The computer readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as Internet transmission media. Thus, the medium may be such a defined and measurable structure including or carrying a signal or information, such as a device carrying a bitstream according to one or more exemplary embodiments. The media may also be a distributed network, so that the computer readable code is stored/transferred and executed in a distributed fashion. Furthermore, the processing element could include a processor or a computer processor, and processing elements may be distributed and/or included in a single device.

According to the exemplary embodiments, even when the relative position between the detection spot from which the biosignal is detected and the source that generates the biosignal is changed, an error according to the change of the relative position may be reduced by converting the biosignal waveform to the reference biosignal waveform. It is possible to receive the information on the biological condition of the subject from the reference biosignal waveform.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A biosignal processing method comprising:
detecting from a first area of a subject a biosignal, which is generated by a movement of a heart existing in a second area of the subject;
generating a biosignal waveform from the biosignal;
determining a relative position of the first area with respect to the second area based on at least one of the biosignal waveform and a direction of the first area; and
converting the biosignal waveform to a reference biosignal waveform based on the relative position.

2. The biosignal processing method of claim 1, wherein the converting comprises:
reading a transfer function corresponding to the relative position from metadata; and
applying the read transfer function to the biosignal waveform to convert the biosignal waveform to the reference biosignal waveform.

3. The biosignal processing method of claim 2, wherein the transfer function comprises a first transfer function of an amplitude part and a second transfer function of a phase part.

4. The biosignal processing method of claim 3, wherein
the first transfer function is defined as an amplitude ratio between biosignal waveforms detected at different positions, and
the second transfer function is defined as a phase difference between the biosignal waveforms detected at the different positions.

5. The biosignal processing method of claim 3, wherein the converting comprises:
dividing the biosignal waveform into an amplitude part and a phase part by using a discrete Fourier transform;
applying the first transfer function to the amplitude part;
applying the second transfer function to the phase part; and
acquiring the reference biosignal waveform by using a discrete Fourier transform.

6. The biosignal processing method of claim 1, wherein the reference biosignal waveform is a biosignal waveform at a reference position.

7. The biosignal processing method of claim 1, wherein the reference position is a position at which heights of the first area and the second area are equal to each other.

8. The biosignal processing method of claim 1, wherein the biosignal is a photoplethysmography signal.

9. The biosignal processing method of claim 1, wherein the direction of the first area is detected by a direction sensor disposed in the first area.

10. The biosignal processing method of claim 9, wherein the direction sensor is a tilt sensor.

11. The biosignal processing method of claim 1, wherein the determining the relative position comprises, when a single relative position is expected from the direction of the first area, determining the expected relative position as the relative position.

12. The biosignal processing method of claim 1, wherein the determining the relative position comprises, when a plurality of relative positions are expected from the direction of the first area, determining one of the plurality of expected relative positions as the relative position.

13. The biosignal processing method of claim 12, wherein the determining one of the plurality of expected relative positions as the relative position comprises:
extracting factors including at least two of an augmentation index, a minimum systolic time, and a reflect wave time; and
comparing the extracted factors with factors corresponding to the reference biosignal wave.

14. The biosignal processing method of claim 1, wherein the first area is a wrist of the subject.

15. The biosignal processing method of claim 1, further comprising estimating information on a biological condition of the subject by using the reference biosignal waveform.

16. The biosignal processing method of claim 15, wherein the information on the biological condition of the subject includes at least one of blood pressure information and vascular compliance information.

17. A biosignal processing apparatus comprising:
a first sensor configured to detect a biosignal, which is generated by a movement of a heart existing in a first area of a subject, from a second area of the subject; and
a processor configured to generate a biosignal waveform from the biosignal and convert the biosignal waveform to a reference biosignal waveform based on a relative position of the second area with respect to the first area.

18. The biosignal processing apparatus of claim 17, further comprising a memory configured to store metadata in which a transfer function for converting the biosignal waveform to the reference biosignal waveform is defined for each position,
wherein the processor is further configured to read a transfer function corresponding to the relative position from the memory and acquire the reference biosignal waveform by applying the read transfer function to the biosignal waveform.

19. The biosignal processing apparatus of claim 17, wherein the reference biosignal waveform is a biosignal waveform at a reference position.

20. The biosignal processing apparatus of claim 17, further comprising a second sensor configured to detect a direction of the second area.

21. A method processing of a biosignal measuring device, the method comprising:
detecting a biosignal from a detection point of the subject on which the biosignal measuring device is placed;
generating a biosignal waveform from the biosignal;
determining a relative position of the biosignal measuring device with respect to a reference point of the subject based on a tilt angle of the biosignal measuring device; and
correcting the biosignal waveform based on the relative position.

22. The method of claim 21, wherein the reference point is located at the heart of the subject and the corrected biosignal waveform indicates a blood pressure of the subject.
